(19) United States
(12) Patent Application Publication
(10) Pub. No.: US 2024/0194295 A1
(43) Pub. Date: Jun. 13, 2024

(54) CELLULAR HETEROGENEITY-ADJUSTED CLONAL METHYLATION (CHALM): A METHYLATION QUANTIFICATION METHOD

(71) Applicants: Wei Li, Irvine, CA (US); Jianfeng XU, Irvine, CA (US); David J. TAGGART, Irvine, CA (US); Helio Health Inc., Irvine, CA (US)

(72) Inventors: Wei LI, Irvine, CA (US); Jianfeng XU, Irvine, CA (US); David J. TAGGART, West Lafayette, IN (US)

(21) Appl. No.: 18/555,639

(22) PCT Filed: Apr. 21, 2022
(86) PCT No.: PCT/US2022/025824

§ 371 Date: Oct. 16, 2023

Related U.S. Application Data
(60) Provisional application No. 63/177,903, filed on Apr. 21, 2021.

(51) Int. Cl. G16B 25/10; G16B 20/20; G16B 40/20

(52) U.S. Cl. CPC G16B 25/10; G16B 20/20; G16B 40/20

(57) ABSTRACT

In certain aspects, provided herein are methods and systems for methylation quantification based on a Cellular Het-crogeneity-Adjusted cLonal Methylation (CHALM) quantification methodology described herein. Disclosed herein, in some aspects, are methods for identifying the methylation status of a biomarker in a single cell. In certain aspects, provided herein are methods for generating a methylation profile of a biomarker associated with a tumor species.

### CROSS-REFERENCE TO RELATED APPLICATIONS

This application claims the priority benefit of U.S. Provisional Patent Application No. 63/177,903, filed on Apr. 21, 2021, the disclosure of which is hereby incorporated herein by reference in its entirety.

### TECHNICAL FIELD OF THE INVENTION

The present invention relates generally to methods for the quantification of methylation, in particular, differentially methylated genes that exhibit distinct biological functions. More specifically, the present invention relates to the binary methylation status (methylated or unmethylated) of a genomic locus in a single cell (e.g., represented by one or more sequence reads in bisulfite sequencing data).

### BACKGROUND OF THE DISCLOSURE

DNA methylation within a genomic locus can impact a diverse array of biological functions. For example, promoter DNA methylation is a well-established mechanism of transcription repression, though its global correlation with gene expression is weak. This weak correlation can be attributed to the failure of current methylation quantification methods to consider the heterogeneity among sequenced bulk cells. The poor correlation between promoter methylation and gene expression is due in part to the overly simplistic nature of the traditional DNA methylation quantification method (i.e., it determines just the mean methylation level of every CpG within a promoter) (Schultz, M. D., Schmitz, R. J. & Ecker, J. R. Trends Genet. 28, 583-585, 2012). Thus, a key disadvantage of this traditional method is that it fails to account for heterogeneity among sequenced bulk cells but treats CpGs within or across cells as if they are identical. There is a need in the art for improved methylation quantification techniques to better understand the link between DNA methylation and biological function.

### SUMMARY OF THE INVENTION

In certain aspects, provided is a method for determining a Cellular Heterogeneity-Adjusted clonal Methylation (CHALM) score for a genomic region, the method comprising: receiving sequencing information comprising sequence reads; determining a number of methylated sequence reads associated with the genomic region, or a portion thereof, from the sequencing information, wherein the methylated sequence reads each comprise methylation of at least one qualified CpG site mapped to the genomic region, or the portion thereof; determining a number of unmethylated sequence reads associated with the genomic region, or the portion thereof, from the sequencing information, wherein the unmethylated sequence reads each comprise at least one qualified CpG site mapped to the genomic region, or the portion thereof, and wherein none of the qualified CpG sites of the unmethylated sequence reads are methylated; and determining the CHALM score for the genomic region based on the number of methylated sequence reads associated with the genomic region, or a the portion thereof, divided by the sum of the numbers of methylated sequence reads and unmethylated sequence reads associated with the genomic region, of the portion thereof.

In some embodiments, the qualified CpG site comprises at least one sequence read covering the CpG site from the sequencing information. In some embodiments, the qualified CpG site comprises at least four sequence reads covering the CpG site from the sequencing information. In some embodiments, the method further comprises determining whether a CpG site is a qualified CpG site based on the number of sequence reads covering the CpG site.

In some embodiments, the method further comprises determining, such as identifying, the genomic region.

In some embodiments, the method comprises determining CHALM scores for two or more genomic regions.

In some embodiments, the sequencing information is obtained from a sequencing technique. In some embodiments, the sequencing technique is a next generation sequencing technique. In some embodiments, the sequencing technique is a whole-genome sequencing technique. In some embodiments, the sequencing technique is a targeted sequencing technique. In some embodiments, the method further comprises performing the sequencing technique. In some embodiments, the sequencing technique comprises sequencing of nucleic acids obtained from a sample from an individual.

In some embodiments, the sample is a blood sample comprising cell-free DNA. In some embodiments, the nucleic acids obtained from the sample are subjected to processing prior to sequencing, wherein the processing enables determination of a methylation status of one or more CpG sites of the nucleic acids. In some embodiments, the processing is an enzyme-based technique for the conversion of unmethylated cytosines to enable the determination of the methylation status of one or more CpG sites. In some embodiments, the enzyme-based technique is an EM-seq technique. In some embodiments, the processing is a bisulfite-based technique.

In some embodiments, the sequence technique is capable of providing paired-end sequencing reads. In some embodiments, the sequencing technique is performed such that the sequencing depth is at least about 50×.

In some embodiments, the received sequencing information is subjected to informatics pre-processing prior to determining the number of methylated and/or unmethylated sequence reads. In some embodiments, the informatics pre-processing comprises removing low-quality reads. In some embodiments, the informatics pre-processing comprises removing sequence adaptor sequences. In some embodiments, the informatics pre-processing comprises mapping sequence reads to a reference genome. In some embodiments, the reference genome is a human reference genome.

In some embodiments, the method further comprises determining differential methylation associated with the genomic region, or the portion thereof, based on the CHALM score for the genomic region. In some embodiments, the differential methylation is determined based on a beta-binomial model.

In some embodiments, the method further comprises correlating the CHALM score for the genomic region with a level of expression of an associated gene.

In some embodiments, the method further comprises correlating the CHALM score for the genomic region with an associated H3K4me3 level.

In other aspects, provided herein is a method of generating a methylation profile of one or more biomarkers from a sample from an individual, wherein the one or more biomarkers comprise one or more genomic regions, the method comprising: determining a CHALM score for each of the one or more genomic regions according to any method described herein; and generating a methylation profile based on the determined CHALM score(s). In some embodiments, the method further comprises determining differential methylation of the one or more genomic regions based on the associated CHALM score.

In some embodiments, the sample is a cfDNA sample. In some embodiments, the individual is suspected of having a cancer. In some embodiments, the cancer is a liver cancer. In some embodiments, the cancer is a colon cancer. In some embodiments, the methylation profile is indicative of the individual having the cancer.

In some embodiments, the method is performed on a system comprising one or more processors, memory storing one or more programs, the one or more programs configured to be executed by the one or more processors, and the one or more programs including instructions for performing a CHALM quantification method as described herein.

In some embodiments, the genomic region is a promoter, or a portion thereof. In some embodiments, the genomic region comprises 10,000 or fewer base pairs.

In other aspects, provided is a system for determining a Cellular Heterogeneity-Adjusted clonal Methylation (CHALM) score for a genomic region, the system comprising: one or more processors; and memory storing one or more programs, the one or more programs configured to be executed by the one or more processors, the one or more programs including instructions for: receiving sequencing information comprising sequence reads; determining a number of methylated sequence reads associated with the genomic region, or a portion thereof, from the sequencing information, wherein the methylated sequence reads each comprise methylation of at least one qualified CpG site mapped to the genomic region, or the portion thereof; determining a number of unmethylated sequence reads associated with the genomic region, or the portion thereof, from the sequencing information, wherein the unmethylated sequence reads each comprise at least one qualified CpG site mapped to the genomic region, or the portion thereof, and wherein none of the qualified CpG sites are methylated; and determining a CHALM score for the genomic region based on the number of methylated sequence reads associated with the genomic region, or a the portion thereof, divided by the sum of the numbers of methylated sequence reads and unmethylated sequence reads associated with the genomic region, of the portion thereof.

In some embodiments, the one or more programs further include instructions for determining differential methylation of the genomic region. In some embodiments, differential methylation is determined based on a beta-binomial model. In some embodiments, the system comprises one or more machine learning classifiers, wherein at least one of the one or more machine learning classifiers comprises the beta-binomial model. In some embodiments, the genomic region is a promoter, or a portion thereof. In some embodiments, the genomic region comprises 10,000 or fewer base pairs.

In certain aspects, provided herein are methods for analyzing the methylation status of cytosines in genomic DNA. In some embodiments, provided is a method for determining a cancer, such as a liver cancer, in an individual. Also provided herein are methods for determining the prognosis of a subject having liver cancer. Further provided herein, in some aspects, are methods that improve the prediction of transcription activities by examining its correlation with gene expression and H3K4me3 level. H3K4me3 is an epigenetic modification to the DNA packaging protein Histone H3 that is associated with transcriptionally active genes.

The subject methods may be employed to diagnose cancer, for example. In particular embodiments, the subject methods may be employed to identify more accurate differentially methylated genes that exhibit distinct biological functions than the traditional methods.

In certain embodiments, provided herein is a method includes a step of “determining the DNA methylation status” of a multitude of independent genomic CpG positions in a biological sample obtained from a patient. Determination of the methylation status may be performed using any method known in the art to be suitable for assessing the methylation of cytosine residues in DNA. Such methods are known in the art and have been described; and one skilled in the art will know how to select the most suitable method depending on the number of samples to be tested, the quantity of sample available, and the like.

In some embodiments, the method quantifies the promoter methylation as the ratio of methylated reads (with ≥1 mCpG) to total reads mapped to a given promoter region.

In some embodiments, the Cellular Heterogeneity-Adjusted clonal Methylation (CHALM)-determined methylation levels exhibit a more linear and monotonic relationship with gene expression.

In some embodiments, the CHALM method provides better prediction of gene expression.

In some embodiments, the CHALM performs best in paired-end and high-depth sequencing dataset.

In some embodiments, the CHALM provides more meaningful results (e.g., a link to biologically relevant function) when compared to traditional methylation quantification methods (e.g., mean methylation level of every CpG within a genomic locus). In some embodiments, the comparing further comprises analyzing traditional methods and the CHALM based on varying definitions of methylated reads

In some embodiments, the method indicates SVD-based imputation method (singular value decomposition (SVD) is not an imputing-algorithm per se) to extend the reads.

In some embodiments, the performance can be improved by extending the reads to different lengths, e.g., up to a length of 300 base pairs.

In some embodiments, the method comprises a sophisticated but intuitive deep learning model.

In some embodiments, the method processes the raw sequencing data into an image-like data structure in which one channel contains methylation information and the other contains read location information.

In some embodiments, the method can leverage more information for gene expression prediction, such as the distance between the read and the transcription start site and the weight of reads with more than one mCpG.

In some embodiments, the method performs better than the traditional methods in terms of predicting gene expression based on promoter CGI methylation levels.

In some embodiments, the CHALM identifies more accurate hypermethylated genes during oncogenesis.

In some embodiments, the CHALM method utilizes an algorithm selected from one or more of the following: a principal component analysis, a logistic regression analysis, a nearest neighbor analysis, a support vector machine, and a neural network model.

In some embodiments, the CHALM provides better correlation between differential methylation and differential gene expression

In some embodiments, the method further identifies de novo differentially methylated regions (DMRs) that are more relevant to the studied underlying mechanisms. The CHALM is a method for quantifying cell heterogeneity-adjusted mean methylation, but it is not a method for quantifying methylation heterogeneity per se.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1a-FIG. 1c illustrate that the CHALM methodology quantifies cell heterogeneity-adjusted DNA methylation level. FIGS. 1a and 1b show two different methylation patterns of a promoter region that cannot be distinguished by the traditional method of promoter methylation analysis. FIG. 1c shows a scatter plot illustrating a comparison of the methylation level calculated by the traditional and CHALM methods for the promoter CGIs of CD3 primary cells.

FIG. 2 shows a deep learning prediction framework. Raw WGBS sequencing reads mapped to a promoter CGI region are processed into an image-like data structure, which has two channels for containing CpG methylation status and the read's distance to the transcription start site. Each row represents one single sequencing read. The image-like data structure is first scanned by different 2D filters for convolution. After three convolution layers and one fully connected layer, a final linear regression layer is used for gene expression prediction.

FIG. 3a-FIG. 3f show the CHALM method better predicts gene expression. FIG. 3a shows scatter plots illustrating the correlation between gene expression and methylation level calculated using both methods. Balanced promoter CGIs (Methods section) of CD3 primary cells are used. Each data point represents the average value of 10 promoter CGIs, and the Spearman correlation is calculated based on original data for each promoter CGI. Comparison of correlation (between the traditional method and CHALM) P values calculated by permutation (Methods section): <1×10−4. FIG. 3b illustrates a similar analysis on low-methylation genes. Comparison of correlation permutation P values: <1×10−4. FIG. 3c shows scatter plots illustrating the correlation between H3K4me3 ChIP-seq intensity and methylation level calculated by the traditional and CHALM methods. Balanced promoter CGIs are used. Comparison of correlation permutation P values: <1×10−4. FIG. 4d illustrates a similar analysis on low-methylation genes. Comparison of correlation permutation P values: <1×10−4. FIGS. 3e and 3f show methylation status of reads mapped to the promoter CGI of HIST2H2BF or SSTR5, respectively. Black circles: mCpG; white circles: CpG.

FIG. 4a-FIG. 4c illustrate that the clonal information is crucial for gene expression prediction. FIG. 4a shows the prediction of gene expression based on raw bisulfite sequencing reads via a deep-learning framework. FIG. 4b shows the disruption of read clonal information by shuffling the mCpGs among mapped reads. FIG. 4c shows the clonal information is disrupted before prediction. Comparison of correlation (between prediction models with and without clonal information disrupted) permutation P values: <1×10−4.

FIG. 5a and FIG. 5b illustrate that the CHALM better identifies hypermethylated promoter CGIs during tumorigenesis. FIG. 5a shows scatter plots illustrating the correlation between differential expression and differential methylation calculated by the traditional and CHALM methods. All promoter CGIs were included for analysis, but only those exhibiting a significant methylation change between normal and cancerous lung tissue were plotted. X-axis: differential methylation ratio; y-axis: differential expression (log 2FoldChange). Comparison of correlation (between the traditional method and CHALM) permutation P values: <1×10−4. FIG. 5 b A large fraction of hypermethylated promoter CGIs identified by the traditional method can be recovered using the CHALM method, as indicated by the Venn diagram. Bar plot shows enrichment of the H3K27me3 peak in three different gene sets.

FIG. 6a-FIG. 6d illustrate that the CHALM provides better identification of functionally related DMRs. FIG. 6a shows KEGG pathway enrichment of the top 2000 hypomethylated DMRs in SCLC. ‘q-value’ refers to one-sided Fisher's Exact test P value adjusted by Benjamini-Hochberg procedure. FIG. 6b shows expression change of genes with hypomethylated DMRs in the KEGG pathways shown in a between LUAD (79) and SCLC (79) patients. The left-to-right order is the same as the top-to-right order shown in FIG. 6a. Two-sided one-sample t-test is used. Sample sizes from left to right for test are 57, 41, 24, 30, and 49, respectively. FIG. 6c shows expression of SSTR1 in LUAD (79) and SCLC (79) patients. Two-sided Wald test P value is adjusted by Benjamini-Hochberg procedure. FIG. 6d shows methylation status of reads mapped to the CHALM-unique hypomethylated DMR found in the SSTR1 promoter region. Only 50 reads are selected for visualization. The methylation levels shown were calculated based on the original dataset. Black circles: mCpG; white circles: CpG. Boxplot definition: line in the box center refers to the median, the limits of box refer to the 25th and 75th percentiles and whiskers are plotted at the highest and lowest points within the 1.5 times interquartile range.

### DETAILED DESCRIPTION OF THE DISCLOSURE

To address pitfalls of traditional promoter methylation quantification methods, a methylation quantification method: Cell Heterogeneity-Adjusted clonal Methylation (CHALM) was developed. The CHALM methodology provides improved prediction of gene expression by interpreting each sequencing read as representing information from a single cell within the sequenced bulk cells. The power of the CHALM methodology in terms of predicting gene expression on a genome-wide scale using a CD3 primary cell dataset was assessed and demonstrated herein. Although the methylation levels calculated by both CHALM and traditional methods were anti-correlated with gene expression, the CHALM-determined methylation levels exhibited a more linear and monotonic relationship with gene expression. Such improvement over a traditional method enables the CHALM methodology provides a significant advancement in the field of methylation analysis and disease detection.

In certain aspects, it was observed that lowly methylated promoter CGIs exhibited a very weak correlation between traditional methylation and gene expression. However, a much stronger correlation between gene expression and CHALM-determined methylation was observed.

DNA methylation is also known to be mutually exclusive with H3K4me3, which is strongly associated with gene expression. Unmethylated H3K4 is capable of releasing the auto-inhibition of DNMT3A by disrupting the interaction between the ATRX-DNMT3-DNMT3L and catalytic domains, thereby inducing de novo methylation (Ooi, S. K. et al. Nature 448, 714-717, 2007).

A negative Spearman correlation between methylation level and H3K4me3 was observed for both the traditional and CHALM methods. However, for genes with low methylation levels, only CHALM-determined methylation was significantly anti-correlated with H3K4me3 level, suggesting that the CHALM method provides a better representation of the mutually exclusive relationship between DNA methylation and H3K4me3.

Additional studies have demonstrated that that CHALM better explains transcription activity.

In some embodiments, the CHALM method exhibited the best correlation with the traditional methylation method. In contrast, the three above-mentioned heterogeneity metrics fit a bell-shaped curve with traditional methylation and thus are not appropriate for direct quantification of methylation, as they cannot distinguish CGIs with low methylation levels (i.e., 0.0-0.2) from those with high methylation levels.

In addition, the CHALM method, which incorporates cell heterogeneity information into DNA methylation quantification, provides a better explanation for the functional consequences of DNA methylation, as evidenced by the demonstrated correlation with gene expression and H3K4me3.

DNA methylation in the promoter region and gene body exhibit different relationships with transcription activity. However, as a causal relationship between gene body methylation and gene expression has not been clearly established, and primarily the focus was on the promoter regions.

Additionally, the importance of clonal information in quantifying DNA methylation using a deep learning model was illustrated and the advantages of the CHALM method for more accurate identification of functionally related DMRs was demonstrated.

Although the definition of CHALM involves the ratio of methylated reads, CHALM is actually intended for quantification of the adjusted methylation level for each CpG site, which makes this method compatible with most existing downstream analysis tools, such as differentially methylated cytosine or DMR calling tools.

When applied to different methylation datasets, the CHALM method enables detection of differentially methylated genes that exhibit distinct biological functions supporting underlying mechanisms.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

A “site” corresponds to a single site, which in some cases is a single base position or a group of correlated base positions, e.g., a CpG site. A “locus” corresponds to a region that includes multiple sites. In some instances, a locus includes one site.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

### Cellular Heterogeneity-Adjusted Clonal Methylation (CHALM) Quantification Methods

In some aspects, provided herein is a Cellular Heterogeneity-Adjusted clonal Methylation (CHALM) quantification method. In some embodiments, the CHALM quantification method is performed to quantify methylation within one or more genomic loci, e.g., a promoter region, such as to assess for biological functions including transcription regulation, gene regulation, and/or gene expression.

In some embodiments, the CHALM quantification method comprises determining the number of methylated reads mapped to a promoter region divided by the sum of the numbers of methylated and unmethylated reads mapped to said promoter region. As described herein, methylated reads comprise at least one methylated CpG site mapped to a promoter region. As described herein, unmethylated reads comprises at least one unmethylated CpG site mapped to the promoter region, wherein all CpG sites mapped to the promoter region are unmethylated. In some embodiments, the reads used in the CHALM quantification method are processed and/or filtered, such as to elongate the read, and/or ensure one or more desired characteristics, e.g., based on read quality (e.g., a Phred score of greater than or equal to 20), read sequencing depth, read length, M-bias, or paired-reads.

In some embodiments, provided is a method for determining a Cellular Heterogeneity-Adjusted clonal Methylation (CHALM) score for a genomic region, the method comprising: receiving sequencing information comprising sequence reads; determining a number of methylated sequence reads associated with the genomic region, or a portion thereof, from the sequencing information, wherein the methylated sequence reads each comprise methylation of at least one qualified CpG site mapped to the genomic region, or the portion thereof; and determining a number of unmethylated sequence reads associated with the genomic region, or the portion thereof, from the sequencing information, wherein the unmethylated sequence reads each comprise at least one qualified CpG site mapped to the genomic region, or the portion thereof, and wherein none of the qualified CpG sites of the unmethylated sequence reads are methylated; and determining the CHALM score for the genomic region based on the number of methylated sequence reads associated with the genomic region, or a the portion thereof, divided by the sum of the numbers of methylated sequence reads and unmethylated sequence reads associated with the genomic region, of the portion thereof.

In some embodiments, the qualified CpG site used to determine a CHALM score is based on the number of sequence reads covering the CpG site, e.g., a CpG site having at least 1, such as at least any of 2, 3, 4, or 5, is considered a qualified CpG site. In some embodiments, the qualified CpG site comprises at least one sequence read covering the CpG site from the sequencing information. In some embodiments, the qualified CpG site comprises at least four sequence reads covering the CpG site from the sequencing information. In some embodiments, the method further comprises determining whether a CpG site is a qualified CpG site based on the number of sequence reads covering the CpG site.

In some embodiments, the method further comprises determining the genomic region, such as a region of the genome that will be evaluated via the CHALM quantification method. In some embodiments, the genomic region is a genomic locus. In some embodiments, the genomic locus comprises one or more desired characteristics, such as size based on base pair, proximity to a gene, known or potential biological implications. In some embodiments, the genomic region comprises, such as is, a promoter region, or a portion thereof. The CHALM quantification methods described herein can be applied to any number of genomic regions. In some embodiments, the method comprises determining CHALM scores for two or more genomic regions. In some embodiments, individual CHALM score are obtained for each genomic region assessed and the separate CHALM score are cumulatively assessed in one or more downstream processes.

In some embodiments, the sequencing information is obtained from a sequencing technique. In some embodiments, the sequencing technique is a next generation sequencing technique. In some embodiments, the sequencing technique is a whole-genome sequencing technique. In some embodiments, the sequencing technique is a targeted sequencing technique. Additional details regarding exemplary sequencing techniques is provided herein.

In some embodiments, the method further comprises performing the sequencing technique.

In some embodiments, the sequencing technique comprises sequencing of nucleic acids obtained from a sample from an individual. In some embodiments, the sample is a blood sample comprising cell-free DNA.

In some embodiments, the nucleic acids obtained from the sample are subjected to processing prior to sequencing, wherein the processing enables determination of a methylation status of one or more CpG sites of the nucleic acids. Exemplary methylation-sensitive sequencing processes and techniques are described herein. In some embodiments, the processing is an enzyme-based technique for the conversion of unmethylated cytosines to enable the determination of the methylation status of one or more CpG sites. In some embodiments, the enzyme-based technique is a non-disruptive sequencing technique, e.g., exhibits reduced DNA damage as compared to certain chemical techniques, such as bisulfite deamination. In some embodiments, the enzyme-based technique is an EM-seq technique.

In some embodiments, the processing is a bisulfite-based technique.

As described herein, certain qualities associated with sequence reads may be used to filter sequence reads used in the CHALM quantification methods taught herein. Such qualities may also guide sequencing techniques used for embodiments of the methods described herein, and may also guide steps for informatics pre-processing of the obtained sequence reads. For example, in some embodiments, the sequence technique is capable of providing paired-end sequencing reads. In some embodiments, the sequencing technique is performed such that the sequencing depth is at least about 50×, such as at least about any of 75×, 100×, 125×, 150×, 175×, 200×, 225×, 250×, 275×, or 300×.

In some embodiments, the received sequencing information is subjected to informatics pre-processing prior to determining the number of methylated and/or unmethylated sequence reads. In some embodiments, the informatics pre-processing comprises removing low-quality reads, e.g., having a Phred score of equal to or greater than 20. In some embodiments, the informatics pre-processing comprises removing sequence adaptor sequences. In some embodiments, the informatics pre-processing comprises removing M-bias. In some embodiments, the informatics pre-processing comprises length filtering, for example, removing sequence reads not satisfying a certain length. In some embodiments, the informatics pre-processing comprises retaining sequencing reads between about 50-300 base pairs. In some embodiments, the informatics pre-processing comprises elongating sequence reads, such as based on mapping to a reference genome. In some embodiments, the elongated sequence reads have an average base pair length of between 50-300 base pairs, such as between any of 100-300 base pairs, 150-300 base pairs, or 150-250 base pairs. In some embodiments, the elongated sequence reads are elongated up to about 300 base pairs.

In some embodiments, the informatics pre-processing comprises mapping sequence reads to a reference genome. In some embodiments, the reference genome is a human reference genome.

In some embodiments, the method further comprises determining differential methylation associated with the genomic region, or the portion thereof, based on the CHALM score for the genomic region. Many differential methylation determination techniques are known in the field, such as techniques involving statistical test for hypothesis testing, e.g., see Shafi et al., Brief Bioinfom, 19, 2018, which is incorporated herein by reference in its entirety. In some embodiments, the differential methylation is determined based on a beta-binomial model. In some embodiments, the differential methylation is determined based on a count-based hypothesis test. In some embodiments, the differential methylation is determined based on a logistic regression-based approach. In some embodiments, the differential methylation is determined based on a Fisher's exact test (FET). In some embodiments, the differential methylation is determined based on a chi-square (χ2) test. In some embodiments, the differential methylation is determined based on one or more regression approaches. In some embodiments, the differential methylation is determined based on a t-test. In some embodiments, the differential methylation is determined based on a moderated t-test. In some embodiments, the differential methylation is determined based on a Goeman's global test. In some embodiments, the differential methylation is determined based on an analysis of variance (ANOVA). In some embodiments, the differential methylation is determined using a machine learning classifier.

In some embodiments, the method further comprises correlating the CHALM score for the genomic region with a level of expression of an associated gene. In some embodiments, the method further comprises obtaining, such as measuring, the level of expression of the associated gene.

In some embodiments, the method further comprises correlating the CHALM score for the genomic region with an associated H3K4me3 level. In some embodiments, the method further comprises obtaining, such as measuring, the H3K4me3 level.

In some aspects, provided is a method of generating a methylation profile of one or more biomarkers from a sample from an individual, wherein the one or more biomarkers comprise one or more genomic regions, the method comprising: determining a CHALM score for each of the one or more genomic regions according to the description provided herein; and generating a methylation profile based on the determined CHALM score(s). In some embodiments, the method further comprises determining differential methylation of the one or more genomic regions based on the associated CHALM score, such as using one or more machine learning classifiers.

In some embodiments, the sample is a cfDNA sample, such as obtained via a liquid biopsy.

In some embodiments, the individual is suspected of having a cancer. In some embodiments, the cancer is a liver cancer. In some embodiments, the cancer is a colon cancer.

In some embodiments, the CHALM score is indicative of the individual having the cancer. In some embodiments, a plurality of CHALM scores are used to assess an individual for having a cancer.

In some embodiments, the method is performed on a system comprising one or more processors, memory storing one or more programs, the one or more programs configured to be executed by the one or more processors, and the one or more programs including instructions for performing a CHALM quantification method as described herein.

In some embodiments, the genomic region is a promoter, or a portion thereof. In some embodiments, the genomic region comprises 10,000 or fewer base pairs, such as 5,000 or fewer bases, 1,000 or fewer bases, 900 or fewer bases, 800 or fewer bases, 700 or fewer bases, 600 or fewer bases, 500 or fewer bases, 400 or fewer bases, 300 or fewer bases, 200 or fewer bases, or 100 or fewer bases.

In some aspects, provided herein is a system for determining a Cellular Heterogeneity-Adjusted cLonal Methylation (CHALM) score for a genomic region, the system comprising: one or more processors; and memory storing one or more programs, the one or more programs configured to be executed by the one or more processors, the one or more programs including instructions for: receiving sequencing information comprising sequence reads; determining a number of methylated sequence reads associated with the genomic region, or a portion thereof, from the sequencing information, wherein the methylated sequence reads each comprise methylation of at least one qualified CpG site mapped to the genomic region, or the portion thereof; determining a number of unmethylated sequence reads associated with the genomic region, or the portion thereof, from the sequencing information, wherein the unmethylated sequence reads each comprise at least one qualified CpG site mapped to the genomic region, or the portion thereof, and wherein none of the qualified CpG sites are methylated; and determining a CHALM score for the genomic region based on the number of methylated sequence reads associated with the genomic region, or a the portion thereof, divided by the sum of the numbers of methylated sequence reads and unmethylated sequence reads associated with the genomic region, of the portion thereof.

In some embodiments, the one or more programs further include instructions for determining differential methylation of the genomic region. In some embodiments, the differential methylation is determined based on a beta-binomial model. In some embodiments, the system comprises one or more machine learning classifiers, wherein at least one of the one or more machine learning classifiers is configured to determine differential methylation based on one or more CHALM scores. In some embodiments, the one or more machine learning classifiers comprises a beta-binomial model.

In some embodiments, the reads used in the CHALM quantification method comprise reads with at least one CpG site mapped to a promoter region, e.g., a read having at least one methylated CpG site in a promoter region.

In some embodiments, the method comprises obtaining, such as measuring or receiving, a plurality of reads. In some embodiments, the method comprises mapping the plurality of reads to a reference genome, such as using BSMAP, e.g., v2.90, or TopHat, e.g., v2.1.0. In some embodiments, the reference genome is a human reference genome or a portion thereof. In some embodiments, the method comprises identifying a promoter region and CpG sites therein, including, e.g., determining a start point and an end point of the promoter region, or a portion thereof, for use in a CHALM quantification method. In some embodiments, the method comprises determining the number of reads having at least one methylated CpG site within a promoter region (methylated reads). In some embodiments, the method comprises determining the number of reads having at least one unmethylated CpG site within a promoter region, wherein all CpG sites of the promoter region of each read are unmethylated (unmethylated reads). In some embodiments, the reads are obtained from paired-end sequencing reads. In some embodiments, the reads are obtained from high-depth sequencing, such as performed at a depth of at least about 50×, such as at least about any of 75×, 100×125×, 150×, 200×, 250×, or 300×. In some embodiments, the reads are obtained from a paired-end, high-depth (such as at least about 50×) sequencing. In some embodiments, the average length of reads is at least about 150 base pairs, such as at least about any of 175 base pairs, 200 base pairs, 225 base pairs, 250 base pairs, 275 base pairs, or 300 base pairs.

In some embodiments, the CHALM quantification method comprises using a CpG site having at least 2 reads, such as at least any of 3, 4, or 5 reads, covering the CpG site. In some embodiments, the CHALM quantification method comprises removing a CpG site having less than 2 reads covering the CpG site from use in the method.

In some embodiments, the CHALM quantification method comprises use one or more promoter regions having a CpG-island, i.e., a CGI promoter. In some embodiments, the CGI promoter comprise one or more CpG-island overlapping with a 2-kb window centered on a gene transcription starting point.

In some embodiments, the method comprises one or more preprocessing techniques. In some embodiments, the preprocessing technique comprises trimming low-quality bases, such as using Trimmomatic, e.g., v0.35. In some embodiments, the preprocessing technique comprises trimming sequencing adaptors, such as using Trimmomatic, e.g., v0.35. In some embodiments, the preprocessing technique comprises trimming low-quality bases and sequencing adaptors, such as using Trimmomatic, e.g., v0.35.

In some embodiments, the CHALM quantification method further comprises determining differential methylation, such as using a beta-binomial model. In some embodiments, differential methylation comprises applying a threshold to determine the significance of the differential methylation. In some embodiments, the threshold for significant differential methylation obtained via a beta-binomial model is about 0.1 (e.g., values equal to or greater than 0.1 are significant).

In certain aspects, the methods provided herein involve non-disruptive methylation sequencing techniques, and/or use of data obtained therefrom. In some embodiments, the non-disruptive methylation sequencing technique is configured to produce sequencing information, such as sequencing reads, suitable for use in determining one or more CHALM scores. In some embodiments, the non-disruptive methylation sequencing technique comprises use of an enzyme to convert a nucleic acid base such that it can be distinguished from sequencing information, such as via deamination of an unmethylated cytosine to a uracil.

In some embodiments, the methods provided herein further comprise performing the non-disruptive methylation sequencing technique. In some embodiments, the non-disruptive methylation sequencing technique is an enzymatic methyl-seq (EM-seq) technique. In some embodiments, the non-disruptive methylation sequencing technique comprises: (a) enzymatically modifying methylated cytosines (such as 5-methylcytosine (5 mc) and 5-hydroxymethylcytosine (5 hmC)) to prevent deamination in further enzymatic steps; (b) enzymatically converting unmethylated cytosines to uracils; (c) performing PCR amplification (thereby converting uracils to thymines; and (d) sequencing using a next generation sequencing technique. Various techniques for performing a non-disruptive methylation sequencing technique have been described in the art. See, e.g., Vaisvila et al., Genome Res, 31, 2021, which is incorporated herein in its entirety. In some embodiments, enzymatically modifying methylated cytosines is performed using TET2 and/or T4-BGT. In some embodiments, the non-disruptive methylation sequencing technique comprises enzymatically converting unmethylated cytosines to uracil using APOBEC3A. In some embodiments, the non-disruptive methylation sequencing technique comprises subjecting a sample comprising genomic DNA, such as a cfDNA sample, to a next generation sequencing library preparation technique. In some embodiments, the next generation sequencing library preparation technique comprises shearing the genomic DNA, such as to obtain a DNA size of less than about 500 base pairs, such as less than about any of 450 base pairs, 400 base pairs, 350 base pairs, or 300 base pairs. In some embodiments, the next generation sequencing library preparation technique comprises a step of end prep of sheared DNA. In some embodiments, the next generation sequencing library preparation technique comprises a step of adaptor ligation. In some embodiments, the next generation sequencing library preparation technique comprises a step of cleaning up adaptor ligated DNA. In some embodiments, the cleaned and ligated DNA is subjected to oxidative enzymes, such as TET2 and/or T4-BGT, to modify methylated cytosines (5-methylcytosines and 5-hydroxymethylcytosines). In some embodiments, the next generation sequencing library preparation technique comprises a step of cleaning enzyme oxidized DNA. In some embodiments, the oxidized DNA is further subjected to enzymatic cytosine deamination (such as using APOBEC3A). In some embodiments, the next generation sequencing library preparation technique comprises a step of PCR amplification of the deaminated DNA. In some embodiments, the next generation sequencing library preparation technique comprises a step of sequencing and quantification. In some embodiments, the method comprises adding a control to the sample comprising genomic DNA, e.g., prior to performing any enzymatic conversion steps.

In some embodiments, the non-disruptive methylation sequencing technique is performed based on targeted genetic locations. In some embodiments, the non-disruptive methylation sequencing technique is performed across a whole genome.

In some embodiments, the data obtained from the non-disruptive methylation sequencing technique comprises a plurality of sequence reads. In some embodiments, the non-disruptive methylation sequencing technique is performed to a sequencing depth of about 50× to about 500×. In some embodiments, the non-disruptive methylation sequencing technique is performed to a sequencing depth of at least about 50×, such as at least about any of 75×, 100×, 125×, 150×, 175×, 200×, 225×, 250×, 275×, 300×, 325×, 350×, 375×, 400×, 425×, 450×, 475×, or 500×. In some embodiments, the non-disruptive methylation sequencing technique is performed to a sequencing depth of about any of 50×, 75×, 100×, 125×, 150×, 175×, 200×, 225×, 250×, 275×, 300×, 325×, 350×, 375×, 400×, 425×, 450×, 475×, or 500×.

In some embodiments, the method further comprises processing the plurality of sequence reads to remove low-quality reads and/or remove adaptor contamination and/or filter based on sequence read size (such as to an average sequence read size of great than about 200 bp). In some embodiments, the method further comprises aligning the plurality of sequence reads with a reference genome, such as a human reference genome.

In some embodiments, the methods provided herein involve non-disruptive methylation sequencing techniques in combination with one or more additional sequencing techniques. In some embodiments, the one or more additional sequencing techniques comprise next-generation sequencing, such as deep sequencing, droplet digital PCR, and/or pyrosequencing. In some embodiments, the sequencing investigates DNA mutations (e.g., cfDNA mutations), RNA, micoRNA, or any combination thereof. For example, the method may comprise performing the non-disruptive methylation sequencing and deep sequencing (e.g., to evaluate mutations).

Suitable sequencing techniques useful for non-disruptive methylation sequencing techniques described herein are well known in the art. In some embodiments, such sequencing techniques may involve, e.g., (i) amplification and detection, or (ii) direct detection, by a variety of methods such as (a) PCR (sequence-specific amplification) such as Taqman(R), (b) DNA sequencing of untreated and treated DNA, (c) sequencing by ligation of dye-modified probes (including cyclic ligation and cleavage), (d) pyrosequencing, (e) single-molecule sequencing, (f) mass spectroscopy, or (g) Southern blot analysis.

In some embodiments, restriction enzyme digestion of PCR products amplified from enzymatically-converted DNA may be used, e.g., the method described by Sadri and Hornsby (1996, Nucl. Acids Res. 24:5058-5059), or COBRA (Combined Bisulfite Restriction Analysis) (Xiong and Laird, 1997, Nucleic Acids Res. 25:2532-2534). COBRA analysis is a quantitative methylation assay useful for determining DNA methylation levels at specific gene loci in small amounts of genomic DNA. Briefly, restriction enzyme digestion is used to reveal methylation-dependent sequence differences in PCR products of enzymatically-converted DNA. PCR amplification of the converted DNA is then performed using primers specific for the CpG sites of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels.

In some embodiments, the methylation profile of selected CpG sites is determined using methylation-Specific PCR (MSP). MSP allows for assessing the methylation status of virtually any group of CpG sites within a CpG island, independent of the use of methylation-sensitive restriction enzymes (Herman et al, 1996, Proc. Nat. Acad. Sci. USA, 93, 9821-9826; U.S. Pat. Nos. 5,786,146, 6,017,704, 6,200,756, 6,265,171 (Herman and Baylin); U.S. Pat. Pub. No. 2010/0144836 (Van England et al); which are hereby incorporated by reference in their entirety). Briefly, DNA is enzymatically deaminated to convert unmethylated, but not methylated cytosines to uracil, and subsequently amplified with primers specific for methylated versus unmethylated DNA. In some instances, typical reagents (e.g., as might be found in a typical MSP-based kit) for MSP analysis include, but are not limited to: methylated and unmethylated PCR primers for specific gene (or methylation-altered DNA sequence or CpG island), optimized PCR buffers and deoxynucleotides, and specific probes. One may use quantitative multiplexed methylation specific PCR (QM-PCR), as described by Fackler et al. Fackler et al, 2004, Cancer Res. 64(13) 4442-4452; or Fackler et al, 2006, Clin. Cancer Res. 12(11 Pt 1) 3306-3310.

In some embodiments, the non-disruptive methylation sequencing technique comprises MethyLight and/or Heavy Methyl Methods. The MethyLight and Heavy Methyl assays are a high-throughput quantitative methylation assay that utilizes fluorescence-based real-time PCR (Taq Man(R)) technology that requires no further manipulations after the PCR step (Eads, C. A. et al, 2000, Nucleic Acid Res. 28, e 32; Cottrell et al, 2007, J. Urology 177, 1753, U.S. Pat. No. 6,331,393 (Laird et al), the contents of which are hereby incorporated by reference in their entirety).

In some embodiments, the non-disruptive methylation sequencing technique comprises Ms-SNuPE techniques. The Ms-SNuPE technique is a quantitative method for assessing methylation differences at specific CpG sites based on enzymatic deamination of DNA, followed by single-nucleotide primer extension (Gonzalgo and Jones, 1997, Nucleic Acids Res. 25, 2529-2531).

In some embodiments, provided are methods for quantifying the average methylation density in a target sequence within a population of genomic DNA. In some instances, quantitative amplification methods (e.g., quantitative PCR or quantitative linear amplification) are used. Methods of quantitative amplification are disclosed in, e.g., U.S. Pat. Nos. 6,180,349; 6,033,854; and 5,972,602, as well as in, e.g., DeGraves, et al, 34(1) BIOTECHNIQUES 106-15 (2003); Deiman B, et al., 20(2) MOL. BIOTECHNOL. 163-79 (2002); and Gibson et al, 6 GENOME RESEARCH 995-1001 (1996).

In some embodiments, the methods provided herein comprise a sequence-based analysis. For example, once it is determined that one particular genomic sequence from a sample is hypermethylated or hypomethylated compared to its counterpart, the amount of this genomic sequence can be determined. Subsequently, this amount can be compared to a standard control value and used to determine the present of liver cancer in the sample. In many instances, it is desirable to amplify a nucleic acid sequence using any of several nucleic acid amplification procedures which are well known in the art. Specifically, nucleic acid amplification is the chemical or enzymatic synthesis of nucleic acid copies which contain a sequence that is complementary to a nucleic acid sequence being amplified (template). The methods and kits may use any nucleic acid amplification or detection methods known to one skilled in the art, such as those described in U.S. Pat. No. 5,525,462 (Takarada et al); U.S. Pat. No. 6,114,117 (Hepp et al); U.S. Pat. No. 6,127,120 (Graham et al); U.S. Pat. No. 6,344,317 (Urnovitz); U.S. Pat. No. 6,448,001 (Oku); U.S. Pat. No. 6,528,632 (Catanzariti et al); and PCT Pub. No. WO 2005/111209 (Nakajima et al); all of which are incorporated herein by reference in their entirety.

In some embodiments, the nucleic acids are amplified by PCR amplification using methodologies known to one skilled in the art. One skilled in the art will recognize, however, that amplification can be accomplished by any known method, such as ligase chain reaction (LCR), Q-replicas amplification, rolling circle amplification, transcription amplification, self-sustained sequence replication, nucleic acid sequence-based amplification (NASBA), each of which provides sufficient amplification. Branched-DNA technology is also optionally used to qualitatively demonstrate the presence of a sequence of the technology, which represents a particular methylation pattern, or to quantitatively determine the amount of this particular genomic sequence in a sample. Nolte reviews branched-DNA signal amplification for direct quantitation of nucleic acid sequences in clinical samples (Nolte, 1998, Adv. Clin. Chem. 33:201-235).

The PCR process is well known in the art and include, for example, reverse transcription PCR, ligation mediated PCR, digital PCR (dPCR), or droplet digital PCR (ddPCR). For a review of PCR methods and protocols, see, e.g., Innis et al, eds., PCR Protocols, A Guide to Methods and Application, Academic Press, Inc., San Diego, Calif. 1990; U.S. Pat. No. 4,683,202 (Mullis). PCR reagents and protocols are also available from commercial vendors, such as Roche Molecular Systems. In some instances, PCR is carried out as an automated process with a thermostable enzyme. In this process, the temperature of the reaction mixture is cycled through a denaturing region, a primer annealing region, and an extension reaction region automatically. Machines specifically adapted for this purpose are commercially available.

Suitable next generation sequencing technologies are widely available. Examples include the 454 Life Sciences platform (Roche, Branford, CT) (Margulies et al. 2005 Nature, 437, 376-380); Illumina's Genome Analyzer, GoldenGate Methylation Assay, or Infinium Methylation Assays, i.e., Infinium HumanMethylation 27K BeadArray or VeraCode GoldenGate methylation array (Illumina, San Diego, CA; Bibkova et al, 2006, Genome Res. 16, 383-393; U.S. Pat. Nos. 6,306,597 and 7,598,035 (Macevicz); U.S. Pat. No. 7,232,656 (Balasubramanian et al.)); QX200™ Droplet Digital™ PCR System from Bio-Rad; or DNA Sequencing by Ligation, SOLID System (Applied Biosystems/Life Technologies; U.S. Pat. Nos. 6,797,470, 7,083,917, 7,166,434, 7,320,865, 7,332,285, 7,364,858, and 7,429,453 (Barany et al); the Helicos True Single Molecule DNA sequencing technology (Harris et al, 2008 Science, 320, 106-109; U.S. Pat. Nos. 7,037,687 and 7,645,596 (Williams et al); 7, 169,560 (Lapidus et al); U.S. Pat. No. 7,769,400 (Harris)), the single molecule, real-time (SMRT™) technology of Pacific Biosciences, and sequencing (Soni and Meller, 2007, Clin. Chem. 53, 1996-2001); semiconductor sequencing (Ion Torrent; Personal Genome Machine); DNA nanoball sequencing; sequencing using technology from Dover Systems (Polonator), and technologies that do not require amplification or otherwise transform native DNA prior to sequencing (e.g., Pacific Biosciences and Helicos), such as nanopore-based strategies (e.g., Oxford Nanopore, Genia Technologies, and Nabsys). These systems allow the sequencing of many nucleic acid molecules isolated from a specimen at high orders of multiplexing in a parallel fashion. Each of these platforms allows sequencing of clonally expanded or non-amplified single molecules of nucleic acid fragments. Certain platforms involve, for example, (i) sequencing by ligation of dye-modified probes (including cyclic ligation and cleavage), (ii) pyrosequencing, and (iii) single-molecule sequencing.

In some embodiments, the analyzing described above comprises quantitatively detecting the methylation status of the amplified product. In some cases, the detection comprises a real-time quantitative probe-based PCR or a digital probe-based PCR. In some cases, the detection comprises a real-time quantitative probe-based PCR. In other cases, the detection comprises a digital probe-based PCR, optionally, a digital droplet PCR.

In some embodiments, the sequencing technique comprises a bisulfite sequencing technique, which can be a disruptive sequencing technique as reagents involved with bisulfite sequencing are known to degrade nucleic acids.

In some aspects, provided herein is a method of generating a methylation profile of one or more biomarkers from a sample from an individual, wherein the one or more biomarkers comprise one or more promoter regions, the method comprising: determining a CHALM score for each of the one or more promoter regions according to any method described herein; and generating a methylation profile based on the determined CHALM score(s). In some embodiments, the method further comprises determining differential methylation of the one or more promoter regions based on the associated CHALM score. In some embodiments, the sample is a cfDNA sample. In some embodiments, the individual is suspected of having a cancer. In some embodiments, the cancer is a liver cancer. In some embodiments, the cancer is a colon cancer. In some embodiments, the methylation profile is indicative of the individual having the cancer.

### Example 1

### Data Analysis Methods

### Methods of RNA-Seq Analysis

Disclosed herein, in certain embodiments, are methods of RNA-seq analysis. Raw sequencing data of CD3 primary cells (GSM1220574), CD14 primary cells (GSM1220575), cancerous and normal lung tissue (GSE70091), and small-cell lung cancer (SCLC, GSE60052) were downloaded from Gene Expression Omnibus (GEO). Raw sequencing data of lung adenocarcinoma (LUAD) samples were downloaded from GDC legacy archive. We used Trimmomatic (0.35)38 to trim low-quality bases and sequencing adapters. TopHat (2.1.0)39 was then used to align sequencing reads to the hg19 human reference genome with default parameters. The hg19 GTF annotation file for transcriptome alignment was downloaded from UCSC annotation database. We used Cufflinks (2.2.1)40 to calculate Fragments Per Kilobase of transcript per Million mapped reads (FPKM) for annotated transcripts. As for differential expression analysis, read counts of transcripts were first calculated by HTSeq (htseq-count, 2.7)41. DEseq2 (1.20)42 was then used to calculate the expression difference and the statistical importance.

### Methods of WGBS Data Pre-Processing

In some embodiments, disclosed herein include a method of determining the WGBS data pre-processing. Raw bisulfite sequencing data of CD3 primary cells (GSM1 186660), CD4 primary cells (GSM1186661), cancerous and normal lung tissue (GSE70091), and LUAD and SCLC (GSE52271) were downloaded from GEO. After trimming low-quality bases and sequencing adapters, we used BSMAP (2.90)43 to align reads to hg19 human reference genome with default parameters. The methratio.py (from BSMAP package) script was then used to calculate the methylation ratios of CpG sites. Only CpG sites covered by at least 4 reads are retained for the downstream analyses.

### Quantifying the Methylation Levels of Promoter CGIs

In some instances, the aforementioned traditional method for calculating promoter methylation level mainly refers to the mean methylation level, which is computed as

$$1 / n {\sum }_{i = 1}^{n} {C}_{i} / ( {C}_{i} + {T}_{i} ) \qquad ( 1 )$$

where Cᵢ, Tᵢ are the counts of methylated cytosine and unmethylated cytosine on the CpG i of the promoter, respectively.

In our work, we also discussed another traditional method, i.e. weighted methylated level, which is computed as

$${\sum }_{i = 1}^{n} {C}_{i} / {\sum }_{i = 1}^{n} {C}_{i} + {T}_{i} \qquad ( 2 )$$

where Cᵢ, Tᵢ are the counts of methylated cytosine and unmethylated cytosine on the CpG i of the promoter, respectively.

The CHALM methylation level is computed as

nₘ/(nₘ+nᵤ) (3)

where nₘ, nᵤ are the counts of methylated reads and unmethylated reads mapped to the promoter regions, respectively. Reads with at least one mCpG site are defined as methylated reads.

### Differentially Methylated Regions (Pre-Defined Regions)

In some embodiments, disclosed herein include traditional method, differential methylation of promoter CGIs were calculated by Metilene (‘pre-defined regions’ mode, 0.2-7) with default parameters.

For CHALM, differential methylation of promoter CGIs were calculated based on beta-binomial model. For a promoter CGI i, we denoted the counts of methylated reads, the counts of unmethylated reads and CHALM methylation ratio as nₘᵢ, nᵤᵢ, pᵢ, respectively. The nₘᵢ and nᵤᵢ are observed values while pi is unknown. Given that sequenced reads are sampled from the sequencing cell population, we used binomial distribution to model the methylated reads

nₘᵢ˜B(nₘᵢ+nᵤᵢ,pᵢ) (4)

where the pⁱ follows a beta distribution beta(αi, βi), which can be estimated by empirical Bayes method. Similar method has already been implemented in our previously published MOABS package. We then repurposed MOABS to calculate the differential CHALM methylation. The cutoff for significant differential methylation: absolute methylation changes are ≥0.1 and FDR adjusted p-value is <0.05.

### Differentially Methylated Regions (Pre-Defined Regions)

In some instances, for traditional method, de novo DMRs are identified by Metilene (‘de novo’ mode, 0.2-7) with default parameters.

For CHALM, we first calculated the CHALM methylation ratio for each CpG site. After reads alignment, we scaned each read for mCpG. If a read had at least one mCpG, other CpG sites on the same read would be treated as mCpG as well.

Then, the CHALM methylation ratio would be calculated with the methratio.py script from BSMAP. CpG sites covered by at least 4 reads were selected for calling de novo DMRs by Metilene (‘de novo’ mode). Identified de novo DMRs by both traditional method and CHALM were annotated to the nearest gene. We then performed pathway enrichment and gene ontology analysis for the differentially methylated genes by using DAVID (6.8) and Enrichr.

### Methods for ChIP-Seq Data Analysis

In some embodiments, H3K4me3 ChIP-seq datasets for CD3 primary cell, CD14 primary cell were downloaded from Roadmap project <https://www.ncbi.nlm.nih. gov/geo/roadmap/epigenomics/?view-matrix>. Sequencing reads were aligned to hg19 human reference by bowtie2 (2.2.7, local mode). We then counted mapped reads for each promoter CGI by htseq-count with default setting. Finally, the H3K4me3 ChIP-seq signal intensity of a promoter CGI was defined as read counts normalized by the length of the promoter CGI.

### Methods to Balance the Promoter CGIs Set

In some embodiments, the promoter CGIs set distribution was adjusted. Since most promoter CGIs are unmethylated, the distribution of methylation value of promoter CGIs is severely biased to 0. In order to balance the distribution, all promoter CGIs (˜12,000) were split into 200 bins based on their traditional methylation value. For each bin, up to 60 promoter CGIs were randomly selected. The final CGIs set (around 3000 promoter CGIs) is composed of the selected promoter CGIs from 200 bins.

### Permutation Test for Comparing Two Correlation Coefficients

In some embodiments, two samples, which have the same size and are used to calculate two Spearman correlation coefficients, r1 and r2, are first pooled into a single sample. In the b-th permutation run, we randomly divided this pooled sample into two halves, which would be used to compute two permutated Spearman correlation coefficients. Then we calculated the difference. We performed 10,000 independent permutation runs to obtain 10,000 differences under the null hypothesis that the two samples are from the same distribution.

### Missing Value Imputation

In some embodiments, missing value was imputed. Since the length of most public bisulfite sequencing datasets is ˜100 bp while the length of promoter CGIs ranges from 201 bp to several kb, a single read can only capture a small proportion of CpG sites of a promoter CGI. In order to rescue the information from the uncaptured CpG sites, low-rank SVD approximation (estimated by the EM algorithm) was used to extend the read based on the information of nearby reads 17. Promoter CGIs larger than 500 bp and with more than 300 mapped reads were selected for imputation. Mapped reads of a promoter CGI were converted into a matrix with column representing CpG sites of this promoter CGI and row representing different reads. Each row contained the methylation status (mCpG: 1; CpG: 0) of CpG sites captured by a single read. The methylation status of the CpG site uncaptured by reads was label as NA and will be imputed by the ‘impute.svd’ function from bcv package17,18 (1.0.1).

### Methods for Deep Learning Prediction

In some embodiments, promoter CGIs with more than 50 mapped reads were selected for deep learning prediction. The methylation status (mCpG: 1; CpG: 0) and the distance of mapped reads to the TSS would be stored into a 3D array. The 3D array is similar to the data structure for storing the positions and pixel information of an image. The first dimension is for storing the mapped reads, which was sorted by the read's methylation fraction.

fₘ=Nₘ/(Nₘ+Nᵤ)

where Nₘ, Nᵤ refers to the number of methylated CpG and unmethylated CpG on this read, respectively. The length of this dimension is 200. When there were less than 200 mapped reads (Nᵣ<200), pseudo-reads were generated by bootstrapping from actual reads. When there were more than 200 mapped reads (Nᵣ>200), 200*F_{size}(Nᵣ−200<200×F_{size}<Nᵣ) reads were randomly selected. Selected reads were then sorted based on methylation fraction and split into 200 bins, with F_{size} reads in each bin. Finally, a pseudo-read was generated based on the mean value of each bin. N, and F_{size} refer to the number of mapped reads and the size factor, respectively.

The second dimension is to store the methylation status of the CpG sites on the reads. The dimension length is 10, which stores the methylation status of 10 CpG sites from a sequencing read. When there were <10 CpG sites, the methylation status of a read CpG site was expanded to a pseudo-CpG site. When there were more than 10 CpG sites, the methylation levels of adjacent CpG sites were merged

To train this image-like 3D array (200×10×2) data, we built a CNN model with PyTorch (version 1.2). Specifically, the input layer is attached to three sequential Conv2d layers along with RELU activation function. The kernel size of the three Conv2d layers is (5,1), (4,1), and (3,1) respectively. The stride for all Con2d layers is (1,1). Since the second dimension of the input data is small, we did not include pooling layer in our model. The final output layer of this CNN model is a linear regression layer. And in order to prevent overfitting, a dropout layer (p=0.2) was added between the convolution layer and the fully connected layer. We then trained the CNN model using Adam as optimizer and MSELoss as loss function in batches of 32 promoter CGIs.

In order to disrupt the clonal information in the control group, we randomly assigned the mCpGs to mapped reads but kept the total number of mCpGs unchanged. We then sorted the reads based on the methylation fraction to obtain the input matrix, which was used for prediction.

Since most promoter CGIs are unmethylated, the original dataset was downsampled to generate a relatively evenly-distributed dataset (balanced promoter CGI set). Downsampled datasets were then randomly split into training set and test set in a manner of 50-50%. After converting the raw bisulfite sequencing reads into the aforementioned 3D matrix, we trained a convolutional neural network (CNN) model to predict gene expression based on this matrix. The testing set was then used to evaluate the performance of this model.

As a contribution to the community, we also generated a pretrained CNN model by using the RNA-seq and WGBS datasets of 23 different normal tissues from the Roadmap epigenomic project. This pretrained model is ready to use for studying the relationship between DNA methylation and gene expression in other datasets that are of researchers' interest.

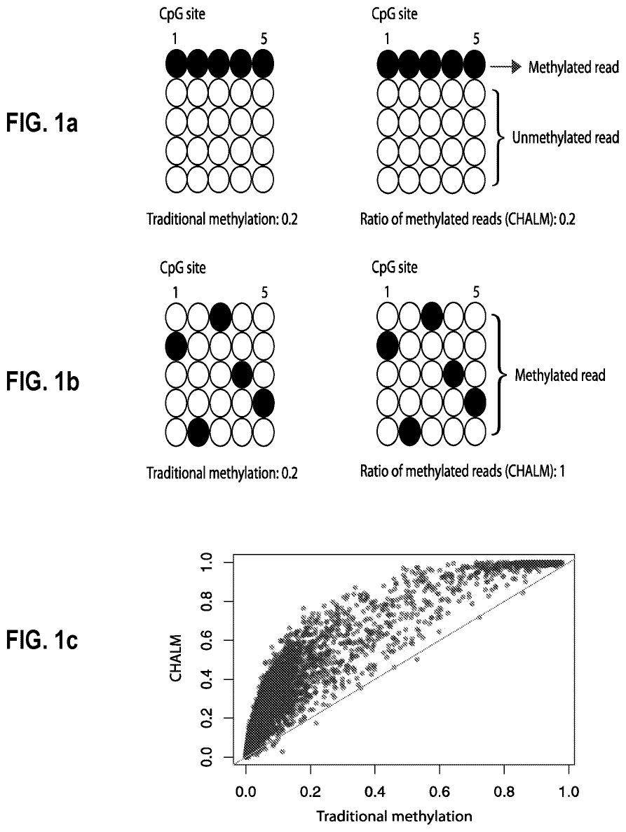

The CHALM method improves the prediction of transcription activities by examining its correlation with gene expression and H3K4me3 level. Further comparisons between CHALM and the traditional method indicate that our method is capable of identifying more accurate differentially methylated genes that exhibit distinct biological functions supporting underlying mechanisms. FIG. 1a-FIG. 1c illustrate that the CHALM methodology quantifies cell heterogeneity-adjusted DNA methylation level. FIGS. 1a and 1b show two different methylation patterns of a promoter region that cannot be distinguished by the traditional method of promoter methylation analysis. FIG. 1c shows a scatter plot illustrating a comparison of the methylation level calculated by the traditional and CHALM methods for the promoter CGIs of CD3 primary cells.

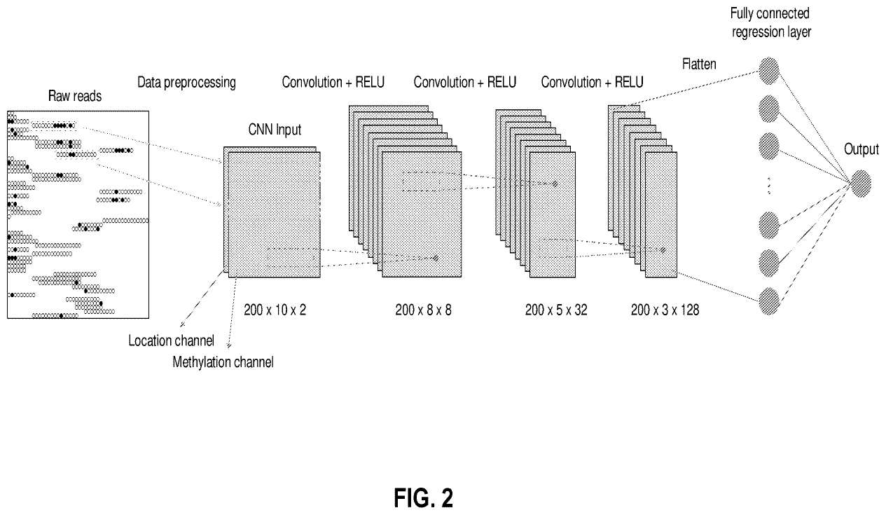

Clonal information is important for gene expression prediction by DNA methylation with a sophisticated but intuitive deep learning model. In order to maximize the amount of useful information extracted from high-throughput sequencing data, the raw sequencing data was processed into an image-like data structure in which one channel contained methylation information and the other contained read location information. FIG. 2 shows a deep learning prediction framework. Raw WGBS sequencing reads mapped to a promoter CGI region are processed into an image-like data structure, which has two channels for containing CpG methylation status and the read's distance to the transcription start site. Each row represents one single sequencing read. The image-like data structure is first scanned by different 2D filters for convolution. After three convolution layers and one fully connected layer, a final linear regression layer is used for gene expression prediction. This deep-learning model outperformed a linear model trained using either traditionally determined or CHALM-determined methylation levels.

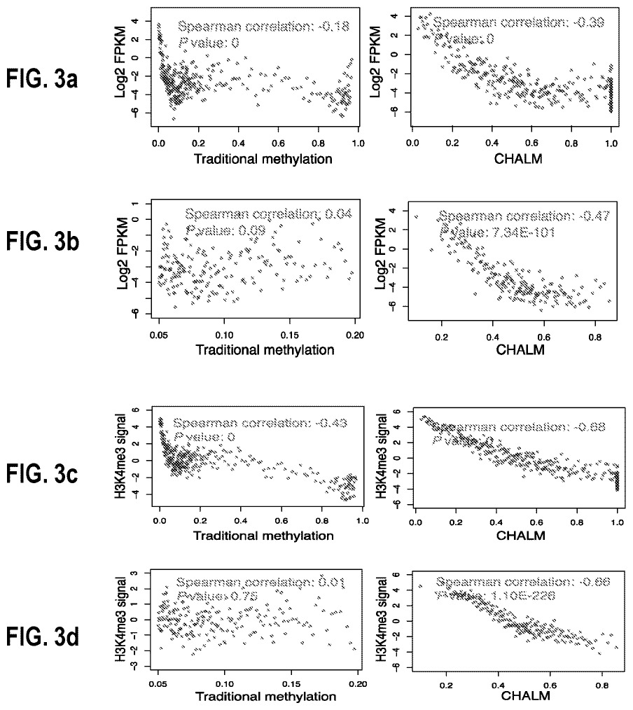

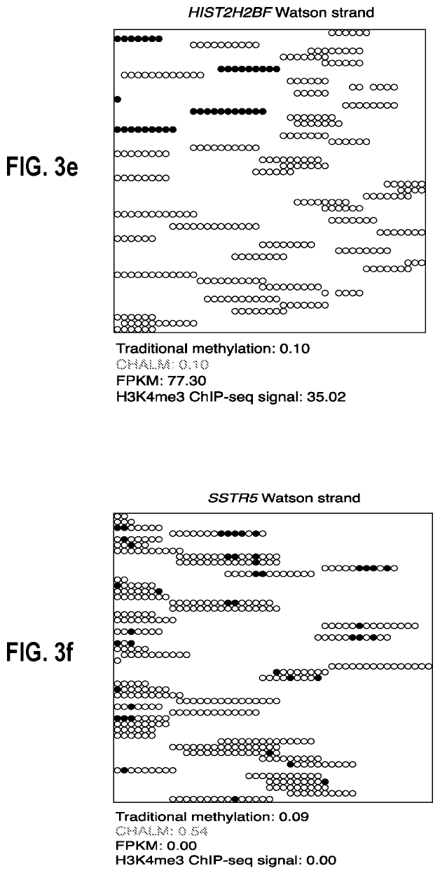

The CHALM method may be evaluated in terms of predicting gene expression on a genome-wide scale using a CD3 primary cell dataset. CHALM better predicts the gene expression and H3K4me3 level in promoter CGIs. FIG. 3a-FIG. 3f show the CHALM method better predicts gene expression. FIG. 3a shows scatter plots illustrating the correlation between gene expression and methylation level calculated using both methods. Balanced promoter CGIs (Methods section) of CD3 primary cells are used. Each data point represents the average value of 10 promoter CGIs, and the Spearman correlation is calculated based on original data for each promoter CGI. Comparison of correlation (between the traditional method and CHALM) P values calculated by permutation (Methods section): <1×10−4. FIG. 3b illustrates a similar analysis on low-methylation genes. Comparison of correlation permutation P values: <1×10−4. FIG. 3c shows scatter plots illustrating the correlation between H3K4me3 ChIP-seq intensity and methylation level calculated by the traditional and CHALM methods. Balanced promoter CGIs are used. Comparison of correlation permutation P values: <1×10−4.

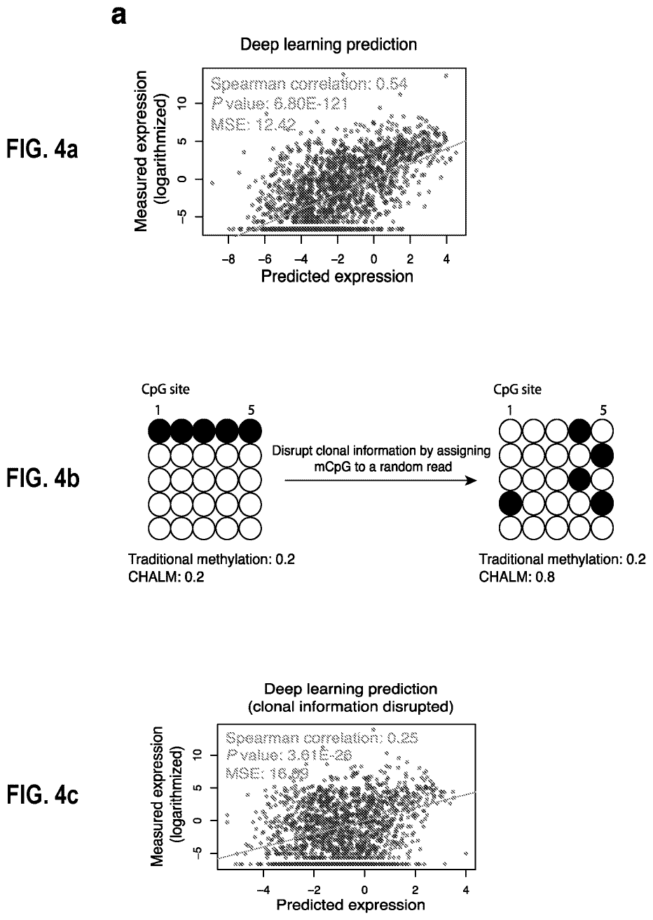

Clonal information is important for gene expression prediction by DNA methylation with a sophisticated but intuitive deep learning model. The data structure leverages more information for gene expression prediction, such as the distance between the read and the transcription start site and the weight of reads with more than one mCpG. These data are then further processed using a convolutional deep neural network for gene expression prediction. As expected, this deep-learning model outperformed a linear model trained using either traditionally determined or CHALM-determined methylation levels. FIG. 4a-FIG. 4c illustrate that the clonal information is crucial for gene expression prediction. FIG. 4a shows the prediction of gene expression based on raw bisulfite sequencing reads via a deep-learning framework. FIG. 4b shows the disruption of read clonal information by shuffling the mCpGs among mapped reads. FIG. 4c shows the clonal information is disrupted before prediction. Comparison of correlation (between prediction models with and without clonal information disrupted) permutation P values: <1×10−4. FIG. 4d illustrates a similar analysis on low-methylation genes. Comparison of correlation permutation P values: <1×10−4. FIGS. 3e and 3f show methylation status of reads mapped to the promoter CGI of HIST2H2BF or SSTR5, respectively. Black circles: mCpG; white circles: CpG.

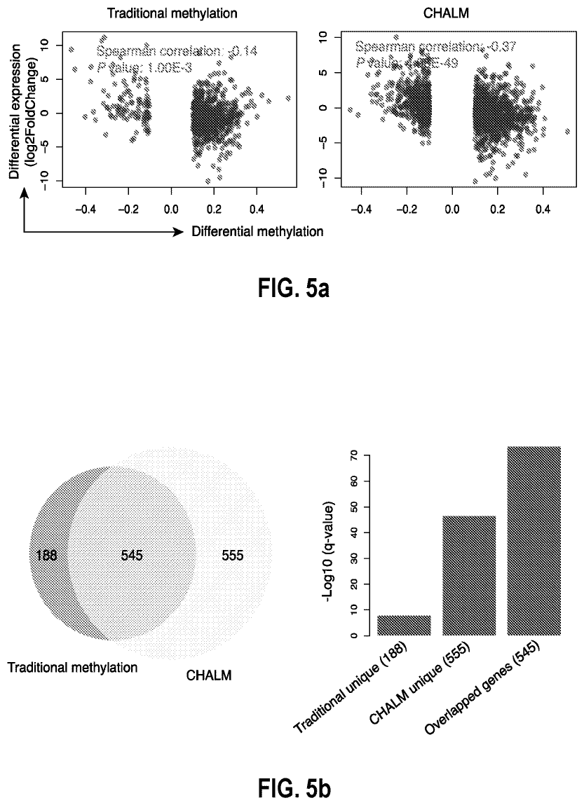

The CHALM method was compared to the traditional method for identifying differentially methylated genes with promoter CGIs in paired cancerous and normal lung tissue samples. The correlation between differential methylation and differential gene expression was significantly greater when the methylation level was calculated using the CHALM method. In addition, the CHALM method not only recovered most of the traditional method-identified hypermethylated genes but also identified a subset of genes that are overlooked by the traditional method. FIG. 5a and FIG. 5b illustrate that the CHALM better identifies hypermethylated promoter CGIs during tumorigenesis. FIG. 5a shows scatter plots illustrating the correlation between differential expression and differential methylation calculated by the traditional and CHALM methods. All promoter CGIs were included for analysis, but only those exhibiting a significant methylation change between normal and cancerous lung tissue were plotted. X-axis: differential methylation ratio; y-axis: differential expression (log 2FoldChange). Comparison of correlation (between the traditional method and CHALM) permutation P values: <1×10−4. FIG. 5 b A large fraction of hypermethylated promoter CGIs identified by the traditional method can be recovered using the CHALM method, as indicated by the Venn diagram. Bar plot shows enrichment of the H3K27me3 peak in three different gene sets.

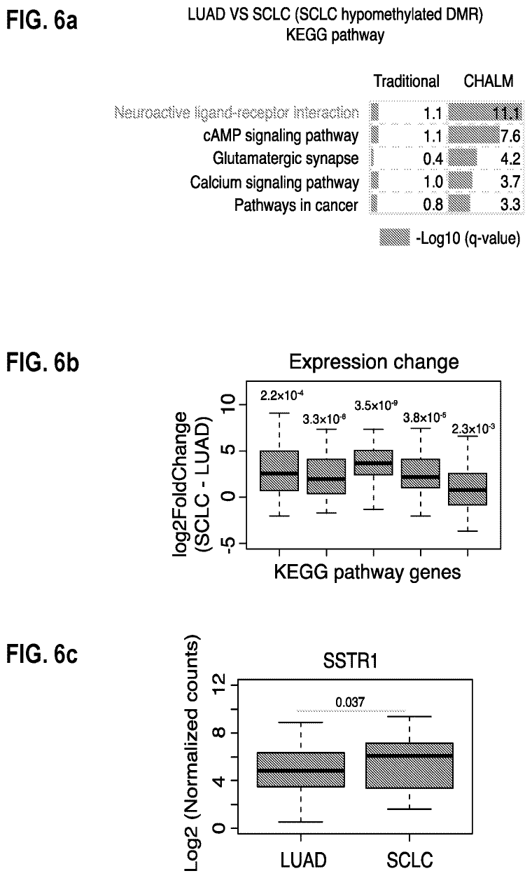

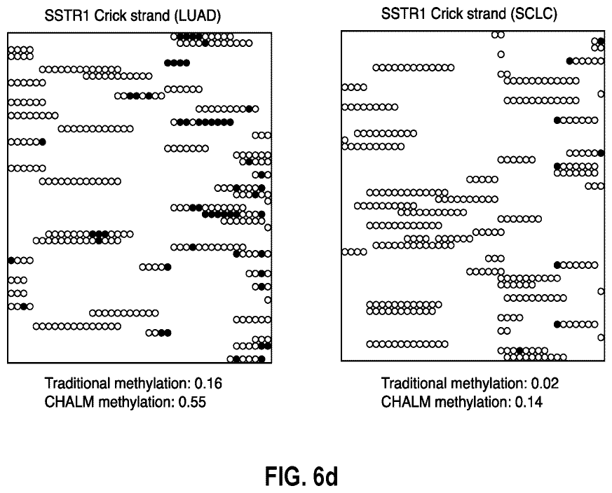

CHALM-determined hypomethylated DMRs in SCLC were more highly enriched in genes of the neuroactive ligand-receptor interaction pathway, which is reportedly activated in SCLC29 (FIG. 6a). Expression of genes from this pathway with hypomethylated DMRs was consistently up-regulated in SCLC. FIG. 6a-FIG. 6d illustrate that the CHALM provides better identification of functionally related DMRs within a genomic locus. FIG. 6a-FIG. 6d are not limited to a promoter region. FIG. 6a shows KEGG pathway enrichment of the top 2000 hypomethylated DMRs in SCLC. ‘q-value’ refers to one-sided Fisher's Exact test P value adjusted by Benjamini-Hochberg procedure. FIG. 6b shows expression change of genes with hypomethylated DMRs in the KEGG pathways shown in a between LUAD (79) and SCLC (79) patients. The left-to-right order is the same as the top-to-right order shown in FIG. 6a. Two-sided one-sample t-test is used. Sample sizes from left to right for test are 57, 41, 24, 30, and 49, respectively. FIG. 6c shows expression of SSTR1 in LUAD (79) and SCLC (79) patients. Two-sided Wald test P value is adjusted by Benjamini-Hochberg procedure. FIG. 6d shows methylation status of reads mapped to the CHALM-unique hypomethylated DMR found in the SSTR1 promoter region. Only 50 reads are selected for visualization. The methylation levels shown were calculated based on the original dataset. Black circles: mCpG; white circles: CpG. Boxplot definition: line in the box center refers to the median, the limits of box refer to the 25th and 75th percentiles and whiskers are plotted at the highest and lowest points within the 1.5 times interquartile range.

What is claimed is:

1. A method for the classification, stratification and/or diagnosis of a tumor species, the method comprising:
(a) providing a tumor-sample to be classified obtained from a tumor of a patient and, optionally, isolating genomic DNA therefrom;
(b) determining a methylation profile from a DNA methylation status of a multitude of independent genomic CpG positions in the genome of said tumor-sample;
(c) classifying the tumor species of the tumor-sample based on the methylation levels as determined in using a classification-rule, wherein the classification-rule is obtained by Cellular Heterogeneity-Adjusted clonal Methylation (CHALM) analysis of a training-data-set, the training-data-set comprising pre-determined methylation data derived from multitude of pre-classified tumor species, wherein said pre-determined methylation data comprises the methylation status of said CpG positions in the genome of each of said pre-classified tumor species;
(d) predicting the gene expression and H3K4me3 level in promoter CGIs;
(e) quantifies the ratio of methylated reads; and
(f) identifying more accurate hypermethylated genes during oncogenesis and de novo DMRs that are more relevant to the studied underlying mechanisms.

2. The method of claim 1, wherein the methylation profile comprises any CpG DNA methylation site.

3. The method of claim 1, wherein the methylation profile comprises one or more biomarkers obtained from whole-genome bisulfite sequencing (WGBS).

4. The method of claim 1, wherein the methylation profile comprises one or more biomarkers obtained from whole-genome enzymatic sequencing.

5. The method of claim 1, wherein determining DNA methylation comprises a bisulfite treatment of the DNA.

6. The method of claim 1, wherein determining DNA methylation comprises an enzymatic conversion of the DNA.

7. The method of claim 1, wherein training of the classification-rule comprises a preceding step of selecting CpG position which of all CpG positions used provide the purest splitting rules, and using said selected CpG positions as a training-data-optimization-set to train the classification-rule.

8. The method of claim 1, wherein training of the classification-rule comprises a step of down-sampling for each tumor species which may include downsampling of the number of boot strap samples to the minority class, the minority class being the lowest sample size of a tumor species in the training-data-set.

9. The method according to any of claims 1 to 6, comprising the further step (a) including the methylation data of the tumor sample as classified in (b) into the training-data-set to obtain an enhanced-training-data-set, and computing an enhanced classification-rule by CHALM analysis based on the enhanced-training-data-set.

10. The computer-implemented method according to any of claims 1 to 7, wherein the methylation data includes for each pre-classified tumor species the methylation status at said CpG position of at least one, two, three, four, five, six or more independent samples.

11. The method of any one of the claims 1 to 8, wherein the biological sample comprises a blood sample.

12. The method of any one of the claims 1-8, wherein the biological sample comprises a tissue biopsy sample.

13. The method of any one of the claims 1-8, wherein the biological sample comprises liquid biopsy sample.

14. A method for determining a Cellular Heterogeneity-Adjusted clonal Methylation (CHALM) score for a genomic region, the method comprising:
receiving sequencing information comprising sequence reads;
determining a number of methylated sequence reads associated with the genomic region, or a portion thereof, from the sequencing information,
wherein the methylated sequence reads each comprise methylation of at least one qualified CpG site mapped to the genomic region, or the portion thereof; and
determining a number of unmethylated sequence reads associated with the genomic region, or the portion thereof, from the sequencing information,
wherein the unmethylated sequence reads each comprise at least one qualified CpG site mapped to the genomic region, or the portion thereof, and wherein none of the qualified CpG sites of the unmethylated sequence reads are methylated;
determining the CHALM score for the genomic region based on the number of methylated sequence reads associated with the genomic region, or a the portion thereof, divided by the sum of the numbers of methylated sequence reads and unmethylated sequence reads associated with the genomic region, of the portion thereof.

15. The method of claim 14, wherein the qualified CpG site comprises at least one sequence read covering the CpG site from the sequencing information.

16. The method of claim 14 or 15, wherein the qualified CpG site comprises at least four sequence reads covering the CpG site from the sequencing information.

17. The method of claim 15 or 16, further comprising determining whether a CpG site is a qualified CpG site based on the number of sequence reads covering the CpG site.

18. The method of any one of claims 14-17, further comprising determining the genomic region.

19. The method of any one of claims 14-18, wherein the method comprises determining CHALM scores for two or more genomic regions.

20. The method of any one of claims 14-19, wherein the sequencing information is obtained from a sequencing technique.

21. The method of claim 20, wherein the sequencing technique is a next generation sequencing technique.

22. The method of claim 20 or 21, wherein the sequencing technique is a whole-genome sequencing technique.

23. The method of claim 20 or 21, wherein the sequencing technique is a targeted sequencing technique.

24. The method of any one of claims 20-23, further comprising performing the sequencing technique.

25. The method of claim 24, wherein the sequencing technique comprises sequencing of nucleic acids obtained from a sample from an individual.

26. The method of claim 25, wherein the sample is a blood sample comprising cell-free DNA.

27. The method of claim 25 or 26, wherein the nucleic acids obtained from the sample are subjected to processing prior to sequencing, wherein the processing enables determination of a methylation status of one or more CpG sites of the nucleic acids.

28. The method of claim 27, wherein the processing is an enzyme-based technique for the conversion of unmethylated cytosines to enable the determination of the methylation status of one or more CpG sites.

29. The method of claim 28, wherein the enzyme-based technique is an EM-seq technique.

30. The method of claim 27, wherein the processing is a bisulfite-based technique.

31. The method of any one of claims 24-30, wherein the sequence technique is capable of providing paired-end sequencing reads.

32. The method of any one of claims 24-30, wherein the sequencing technique is performed such that the sequencing depth is at least about 50×.

33. The method of any one of claims 14-32, wherein the received sequencing information is subjected to informatics pre-processing prior to determining the number of methylated and/or unmethylated sequence reads.

34. The method of claim 33, wherein the informatics pre-processing comprises removing low-quality reads.

35. The method of claim 33 or 34, wherein the informatics pre-processing comprises removing sequence adaptor sequences.

36. The method of any one of claims 33-35, wherein the informatics pre-processing comprises mapping sequence reads to a reference genome.

37. The method of claim 36, wherein the reference genome is a human reference genome.

38. The method of any one of claims 14-37, further comprising determining differential methylation associated with the genomic region, or the portion thereof, based on the CHALM score for the genomic region.

39. The method of claim 38, wherein the differential methylation is determined based on a beta-binomial model.

40. The method of any one of claims 14-39, further comprising correlating the CHALM score for the genomic region with a level of expression of an associated gene.

41. The method of any one of claims 14-40, further comprising correlating the CHALM score for the genomic region with an associated H3K4me3 level.

42. A method of generating a methylation profile of one or more biomarkers from a sample from an individual,
wherein the one or more biomarkers comprise one or more genomic regions, the method comprising:
determining a CHALM score for each of the one or more genomic regions according to claims 14-41; and
generating a methylation profile based on the determined CHALM score(s).

43. The method of claim 41, further comprising determining differential methylation of the one or more genomic regions based on the associated CHALM score.

44. The method of claim 41 or 42, wherein the sample is a cfDNA sample.

45. The method of any one of claims 41-44, wherein the individual is suspected of having a cancer.

46. The method of claim 44, wherein the cancer is a liver cancer.

47. The method of claim 44, wherein the cancer is a colon cancer.

48. The method of any one of claims 45-47, wherein the CHALM score is indicative of the individual having the cancer.

49. The method of claim 14, wherein the method is performed on a system comprising one or more processors.

50. The method of any one of claims 14-49, wherein the genomic region is a promoter, or a portion thereof.

51. The method of any one of claims 14-50, wherein the genomic region comprises 10,000 or fewer base pairs.

52. A system for determining a Cellular Heterogeneity-Adjusted clonal Methylation (CHALM) score for a genomic region, the system comprising:
one or more processors; and
memory storing one or more programs, the one or more programs configured to be executed by the one or more processors, the one or more programs including instructions for:
receiving sequencing information comprising sequence reads;
determining a number of methylated sequence reads associated with the genomic region, or a portion thereof, from the sequencing information,
wherein the methylated sequence reads each comprise methylation of at least one qualified CpG site mapped to the genomic region, or the portion thereof;
determining a number of unmethylated sequence reads associated with the genomic region, or the portion thereof, from the sequencing information,
wherein the unmethylated sequence reads each comprise at least one qualified CpG site mapped to the genomic region, or the portion thereof, and wherein none of the qualified CpG sites are methylated; and
determining a CHALM score for the genomic region based on the number of methylated sequence reads associated with the genomic region, or a the portion thereof, divided by the sum of the numbers of methylated sequence reads and unmethylated sequence reads associated with the genomic region, of the portion thereof.

53. The system of claim 52, wherein the one or more programs further include instructions for determining differential methylation of the genomic region.

54. The system of claim 53, wherein differential methylation is determined based on a beta-binomial model.

55. The system of claim 54 wherein the system comprises one or more machine learning classifiers, wherein at least one of the one or more machine learning classifiers comprises the beta-binomial model.

56. The system of any one of claims 52-55, wherein the genomic region is a promoter, or a portion thereof.

57. The system of any one of claims 52-56, wherein the genomic region comprises 10,000 or fewer base pairs.
